(19) United States
(12) Patent Application Publication
(10) Pub. No.: US 2015/0105689 A1
(43) Pub. Date: Apr. 16, 2015

(54) PATTERN ELECTRORETINOGRAPHY FOR EVALUATING A NEUROLOGICAL CONDITION

(71) Applicant: Robert F. Miller, Eden Prarie, MN (US)

(72) Inventor: Robert F. Miller, Eden Prarie, MN (US)

(21) Appl. No.: 14/511,798

(22) Filed: Oct. 10, 2014

Related U.S. Application Data
(60) Provisional application No. 61/889,269, filed on Oct. 10, 2013.

(51) Int. Cl. A61B 5/0496; A61B 5/16

(52) U.S. Cl. CPC A61B 5/0496; A61B 5/16
USPC 600/558

(57) ABSTRACT

A system includes a display, sensor, and a processor. The display is configured to provide a controlled light stimulus. The sensor has an electrode configured for coupling to tissue. The sensor is configured to generate an output signal corresponding to an electroretinogram based on light evoked activity associated with glutamatergic synaptic processing. The light evoked activity is in response to an image on the display. The processor is coupled to the sensor and is coupled to the display. The processor is configured to classify a neurological condition associated with the tissue. The classification is based on the output signal.

### CLAIM OF PRIORITY

This patent application claims the benefit of priority of U.S. Provisional Patent Application Ser. No. 61/889,269, filed on Oct. 10, 2013, which is hereby incorporated by reference herein in its entirety.

### STATEMENT REGARDING FEDERALLY SPONSORED RESEARCH OR DEVELOPMENT

This invention was made with government support under 1R21 MH100622-01 awarded by the National Institute of Mental Health. The government has certain rights in the invention.

### BACKGROUND

Schizophrenia is a complex disorder that affects about 1% of the global population. The onset of the disease typically occurs in late adolescence/early adulthood. Schizophrenia is often associated with unusual behavior and hallucinations. Traditionally, schizophrenia is diagnosed based on subjective criteria.

### OVERVIEW

The present inventor has recognized the burden associated with reliance on subjective criteria for diagnosing disorders such as schizophrenia. One example of the present subject matter is directed to providing objective criteria by which schizophrenia can be diagnosed. Data for diagnosis is derived from an electroretinogram signal.

The present subject matter includes methods for identifying and studying patients with psychiatric diseases. One example is configured to evaluate schizophrenic patients using an ophthalmological technique referred to as the electroretinogram or ERG. The ERG is an electrical signal non-invasively recorded from the front of the eye.

An example utilizes a variant of the electroretinogram (ERG), called pERG. Pattern ERG reflects activity of retinal ganglion cells and has a signature response component that can be attributed to light-evoked activity of N-methyl-D-Aspartate or NMDA receptors (NMDARs). Abnormal regulation of NMDARs has been implicated as one cause of schizophrenia.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a system diagram, according to one example.

FIG. 2 illustrates a flow chart of a method, according to one example.

FIG. 3 illustrates data for a mouse pERG evoked by shifting checkerboard pattern, according to one example.

FIG. 4 illustrates data for a mouse pERG, according to one example.

FIG. 5 illustrates photopic flash ERG data, according to one example.

FIG. 6 illustrates oscillatory potentials (OP), according to one example.

FIG. 7 illustrates cone flicker responses, according to one example.

FIG. 8 illustrates photopic Negative Response (PhNR), according to one example.

FIG. 9 illustrates a PhNR delay and amplitude differences, according to one example.

FIG. 10 illustrates an example of human pattern Electroretinogram (pERG) raw trace and smoothing filter, according to one example.

FIG. 11 illustrates pERG recordings showing lower amplitude responses for schizophrenic subjects, according to one example.

FIG. 12 illustrates mean and SE for pERG, according to one example.

FIG. 13 illustrates a flow chart of a method, according to one example.

### DETAILED DESCRIPTION

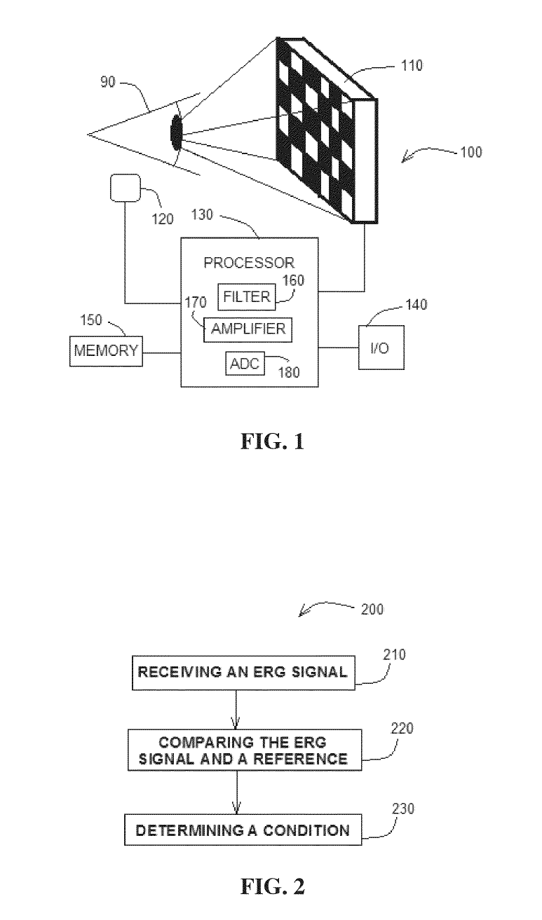

FIG. 1 illustrates a diagram of system 100, according to one example. System 100 includes display 110, sensor 120, processor 130, interface 140, and memory 150. The figure also illustrates eye 90.

Display 110 can include a visual display such as a cathode ray tube (CRT) display, a light emitting diode (LED) display, a plasma display, a liquid crystal display or other device on which a visible image can be depicted. In the example illustrated, display 110 is electrically coupled to processor 130, however, in other examples, display 110 is coupled by a wireless link (such as a radio frequency channel) or a fiber-optic coupling.

Processor 130 can include any combination of electronic circuitry and programming to implement an algorithm as described elsewhere in this document. Processor 130 can include an analog circuit, a digital signal processor, or a computer.

Processor 130 can include any number of modules or circuits configured to perform a particular function. In the example shown, processor 130 can include filter 160. Filter 160 can include a band pass filter, a low pass filter, a high pass filter, or other filter. Processor 130 can include amplifier 170. Amplifier 170 can include a digital or analog circuit. Processor 130 can include analog-to-digital converter (ADC) 180. ADC 180 is configured to receive an analog signal and generate a corresponding digital representation of the signal.

Processor 130 can be coupled to interface 140 and coupled to memory 150. Interface 140 can include a touch screen, a keyboard, a mouse (or other cursor control), a display, a printer, a network interface, or other device. Memory 150 can provide storage for instructions that are executable by processor 130, data for use by processor 130, or other information. In one example, memory 150 provides storage for a reference signal.

Sensor 120 can include a non-invasive contact surface. The contact surface, such as an electrode, can be affixed at a tissue site near an eye of a subject. Sensor 120 can include a plurality of separate electrodes spaced apart from each other and configured in a single unit as illustrated. An electrode can be placed on the cornea, in the vitreous, or inside the retina. In other examples, a plurality of sensors, each as represented by sensor 120, is affixed to tissue of a subject. Sensor 120 is coupled electrically to processor 130. Sensor 120 provides an electrical signal corresponding to a measured parameter detected by the contact surface.

System 100 is configured to provide an electroretionogram. In the example illustrated, sensor 120 is affixed to tissue at a surface near eye 90. An image depicted on display 110 is observed by eye 90. An electrical signal, corresponding to the observed image, is provided by sensor 120. Suitable processing by processor 130 allows interpretation of the signal and provides an indication of the neurological condition of the subject.

FIG. 2 illustrates a flow chart of method 200, according to one example. Method 200, at 210, includes receiving an electroretinogram (ERG) signal from a surface sensor. The ERG signal corresponds to tissue of interest for a subject in response to visible light stimulation. At 220, method 200 includes comparing the ERG signal and a reference. The reference can include stored information derived from the same subject or any number of other subjects. The ERG signal and the reference can be represented in the time domain or in the frequency domain. A processor can be configured to conduct the comparison can and this can include comparing a particular parameter or comparing features. At 230, method 200 includes determining a neurological condition of the subject based on the comparison. In various examples, this can include making a determination concerning schizophrenia or other neurological condition.

The visible stimulation provided to the subject can include a predetermined geometric pattern. For example, the stimulation can include a shifting checkerboard pattern, horizontal bar pattern, or a vertical bar pattern. The processor can be configured to receive and process time domain data. Comparing can include comparing an amplitude. The signal from the sensor can be processed using a filter, an amplifier, or other module. In one example, the processor can be configured to implement independent component analysis (ICA), a neural network, artificial intelligence, or an expert system. The method can be configured to diagnose schizophrenia or other neurological condition.

An electroretionogram, based on an eye of a subject, can provide data as to cerebral function and provide data as to retinal biomarkers of psychopathology.

The onset of schizophrenia typically involves genetic and perinatal insults and at least one additional factor that can be viewed as a functional deficiency in NMDARs.

In the vertebrate retina, the first synapse in the visual pathway in which NMDA receptors are significantly involved is the light-evoked activity of retinal ganglion and amacrine cells.

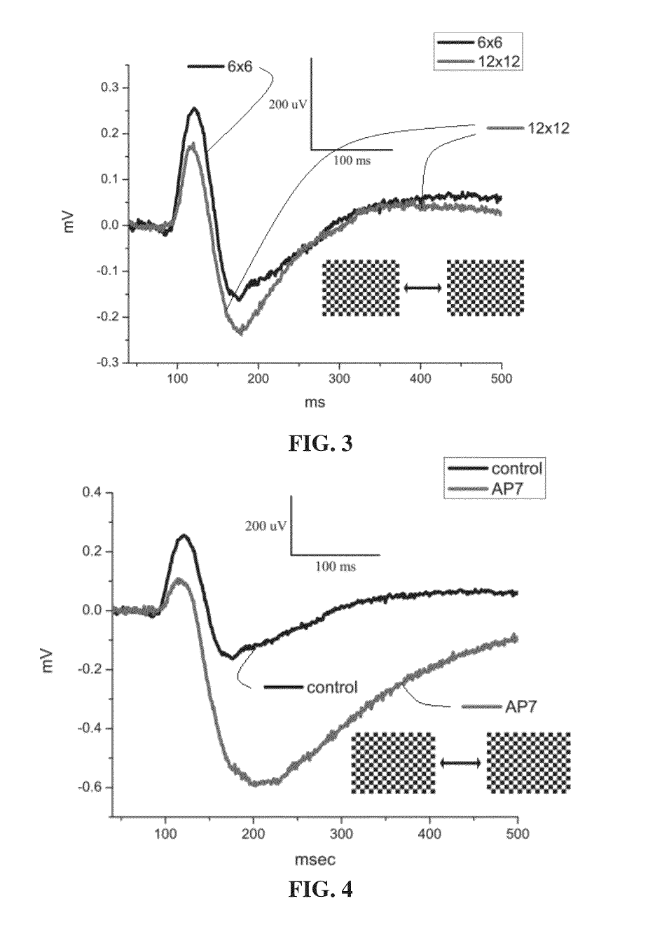

In one example, data is derived from isolated, perfused mouse retina, using a stimulus paradigm similar to that used in humans to evoke the pERG. Pharmacological studies suggest that NMDA receptors provide a major contribution to this response, as shown in FIGS. 3 and 4.

FIG. 3 illustrates data for a mouse pERG evoked by shifting checkerboard pattern at two different spatial frequencies.

FIG. 4 illustrates data for a mouse pERG. Relative to the control, the data for D-AP7 (NMDAR antagonist) illustrates a profound effect on the pERG and significantly reduces the positive response (in vitro).

Control subjects and subjects having a diagnosis of schizophrenia can be used to evaluate the light-evoked ERG responses, using a range of light stimulation and ERG recording strategies.

For example, human subjects can be used to demonstrate that NMDA receptor hypofunction in schizophrenia patients is detectable though measurements of retinal function.

Data corresponding to cognitive functioning (the Dot Pattern Expectancy Task, a task designed to measure context processing, and the Jittered Orientation Visual Integration Task, a task designed to measure visual integration deficits) can be used to evaluate the relationship between cognitive functioning on these tasks and pERG response.

One example of the present subject matter can include using a Diagnosys LLC e³ Clinical ERG testing apparatus, which allows measurement of the pERG, the scotopic ERG, the photopic ERG and the Photopic Negative Response. The pERG can be obtained with the subject facing a monitor screen on which a checkerboard pattern is generated through the software. The size of an individual checkerboard square can subtend 0.8 degrees visual angle. Field width can be 15 degrees horizontally when viewed from 100 cm. White squares of the pattern can have a luminance of 99 cd/m² (candela per square meter), with 100% contrast, reversing at 2.1 reversals/sec. Frequency bandwidth can be 0.625 to 100 Hz and 150 sweeps can be averaged to generate a single pERG result. The room lights can be dimmed during the procedure.

In other examples, light stimuli can include a checkerboard pattern of 0.05 to 0.1 cycles/degree with a diffuse background light of 10 Cd/m²; the checkerboard pattern can have an average intensity of 480 Cd/m².

Subjects diagnosed with schizophrenia can have significant differences in the amplitude of the major pERG components in which the initial negative response (N35) is smaller in schizophrenia subjects compared to controls and this difference is also apparent for the P50 and a bit less so for the N95 component; the photopic flash ERG and Oscillatory Potentials are slower in time course, but similar in amplitude for schizophrenia subjects; the cone flicker response can be both slower and smaller in amplitude when schizophrenia subjects are compared to controls.

The pERG is generally considered to be a response generated by third-order neurons and can be correlated with ganglion cell activity. Some of the differences observed in human schizophrenia and control subjects are similar to that observed in the pERG of the mouse retina, after blocking the light-responses with an NMDAR antagonist (decreased positive response component). Thus, it is possible that the observed differences reflect differences in the degree to which NMDARs contribute to the pERG between the two study groups and are consistent with the NMDAR hypofunction hypothesis of schizophrenia.

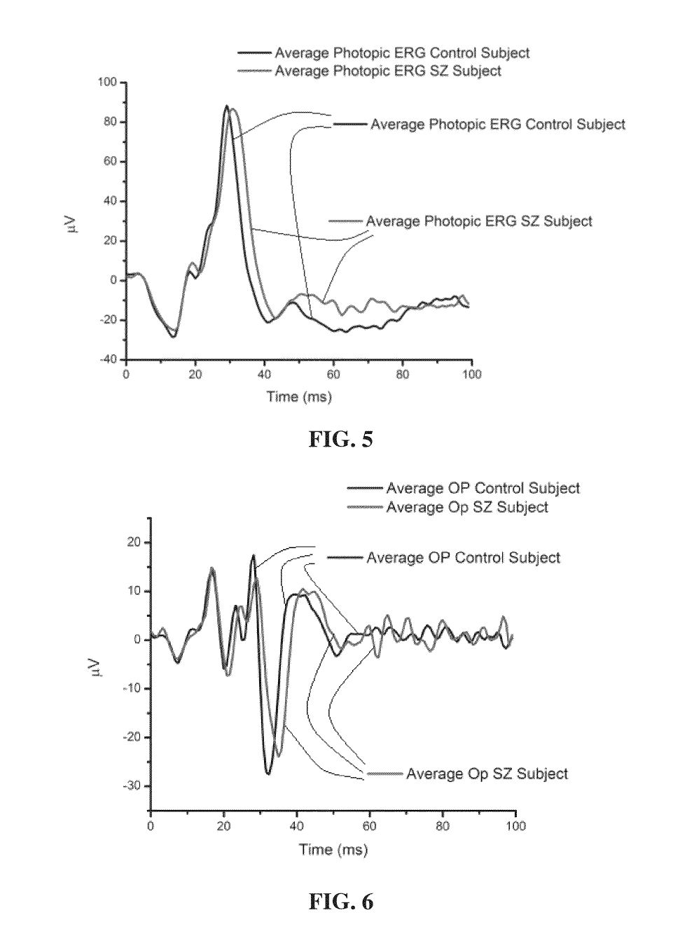

Human data is shown in FIGS. 5-12.

### Photopic ERG

FIG. 5 illustrates photopic ERG (sometimes referred to as photopic flash ERG) data. The figure illustrates that schizophrenia subjects have slower but normal amplitude responses. The data shown represent stimulus frequency of 4 ms flash 1 Hz, 3 Cd·s/m² (candela-second per square meter); background white light 34 cd/m².

The photopic ERG is a cone-dominated response using much brighter light flashes compared to scotopic ERG (described elsewhere in this document) after a period of light adaptation. A significant difference is noted in the latency of the b-wave of the photopic ERG, in which the schizophrenia patient exhibits a slower response compared to a control subject. The magnitude of the difference between the schizophrenia patients and control subjects is similar to that noted in the photopic negative response (described elsewhere in this document).

The results from human subjects show that the pERG responses of subjects with schizophrenia (SZ) differs substantially from those of the normal control subject. The early positive component (P50) is significantly reduced in amplitude and the late negative-going phase of the response (N95) is significantly larger when compared to control responses. Notably, the pERG waveform changes for subjects with schizophrenia closely mirror the changes in the pERG response of mouse retinas in which NMDA receptor activity has been pharmacologically blocked. This similarity suggests that the altered pERG of the schizophrenic subjects may result in part from NMDA receptor hypofunction.

### Oscillatory Potentials (OP)

FIG. 6 illustrates oscillatory potentials (OP). The data shows latency differences between controls and schizophrenia subjects. Recording bandwidth can be between 75 and 300 Hz.

Riding on the ERG is a series of wavelets that can be recorded in isolation using appropriate electronic filtering. Filtering can include using a low pass filter having a cut-off frequency selected to yield a smooth signal. The filter can be an active or passive filter and in one example, is implemented using a digital signal processor. FIG. 6 illustrates the differences in the delay of the ERG compared to the controls.

### Photopic Flicker Response

FIG. 7 illustrates cone flicker responses using intermittent light stimulation of approximately 30 Hz and using a range of stimulus intensities. The light stimulation at 30 Hz is targeted to the cones rather than the rods. The data shows that schizophrenia subjects have lower amplitude and slower responses. Stimulus pulse 4 ms; intensity 3 Cd·s/m²; Background white light, 34 Cd/m².

Unlike the Photopic Negative Response (described elsewhere in this document), the photopic flicker response reveals a longer latency in schizophrenia patients, as well as a depression in the amplitude. FIG. 7 illustrates this difference, comparing 17 controls and 14 schizophrenia patients. With a flicker response, the peak delay time (latency) and the peak amplitude is reduced.

### Photopic Negative Response (PhNR)

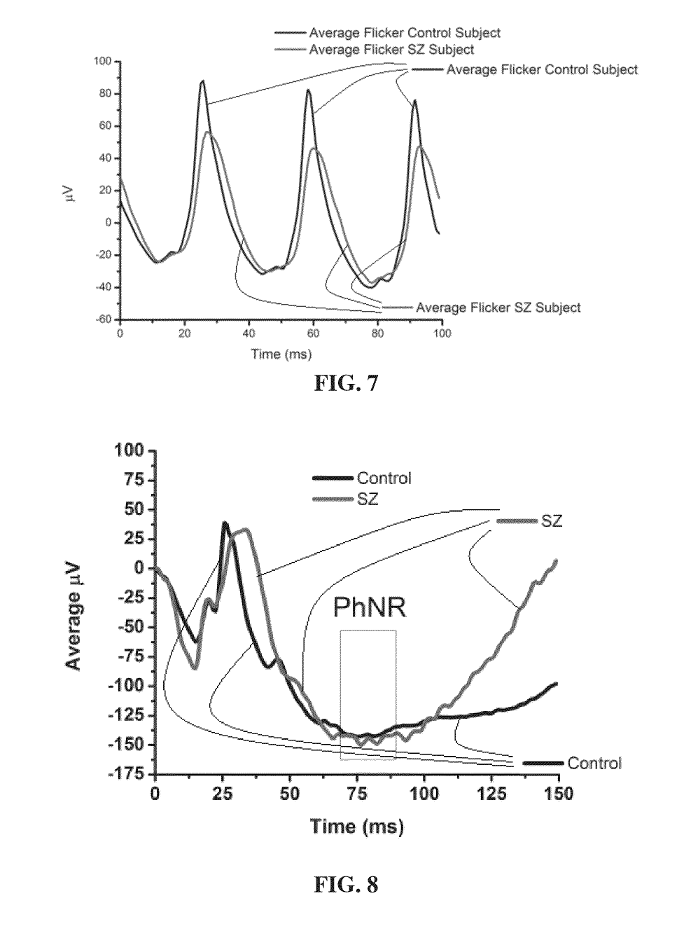

FIG. 8 illustrates photopic Negative Response (PhNR) and shows difference between schizophrenia subjects and controls. This response has been attributed to retinal ganglion cell activity. Stimulation included 4 ms red stimulus 1 Cd·s/m² at 4 Hz on blue background 10 Cd/m²; 50 sweeps/trace.

Photopic negative response data exhibits a late negative response (the negative response occurs after the positive response). In schizophrenia, the negative response may be late or missing.

In one example, the photopic negative response is generated by a stimulus including a red flash (635 nm) on a blue background (450 nm) of 1, 5 and 7 cd·s/m², generated by a full-field Ganzfeld LED-stimulator (Diagnosys LLC, Boston, Mass.). A difference noted between schizophrenics and control subjects relates to the latency of the b-wave. The b-wave is the peak response that occurs at about 25 ms in the example provided in FIG. 8. The difference noted is highly significant with probability values at or below 0.02, pointing to a very significant difference.

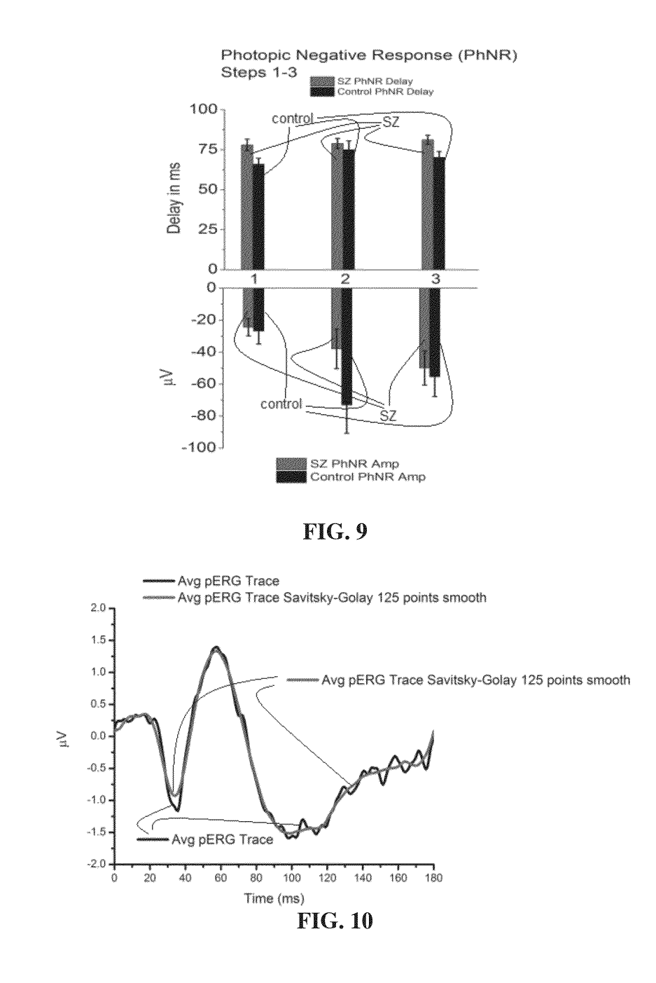

FIG. 9 illustrates the PhNR delay (upper panel, in ms) showed some differences (steps 1 & 3) while amplitude differences (lower panel, in μV) were observed for step 3, but no consistency is noted for all step responses. Stimulus conditions as above with red flash of 1, 5, and 7 Cd·s/m².

A method directed to photopic negative response can include receiving an electroretinogram (ERG) signal corresponding to a subject. The signal is in response to stimulus including red light on a blue background. The signal is based on a sensor coupled to tissue of the subject. The method includes identifying a positive response in the signal. The method also includes detecting absence of a negative response in the signal following the positive response. In response to detecting absence of a negative response, the method includes making a determination concerning a neurological condition. The neurological condition can include schizophrenia.

In the case of schizophrenia, if the stimulation for a photopic negative response has light intensity of 1 Cd·s/m², the negative response can be small and for higher intensity, such as 5 or 7 Cd·s/m², then the negative response can be missing. Detecting the missing negative response includes monitoring for an amplitude of the ERG signal.

pERG

FIG. 10 illustrates an example of human pERG raw trace and smoothing filter based on Savitsky-Golay algorithm. Stimulus conditions are described elsewhere in this document.

The pattern ERG is generated by having the subjects look at a checkerboard pattern and shift the pattern by one square every two seconds. This stimulus does not generate any change in luminance. The stimulus pattern is projected onto the fovea to obtain large amplitude signals.

Two methods can be used to generate the pERG. In a first method, a checkerboard pattern is presented to the subject using a conventional CRT (cathode ray tube) display. The data is recorded from the eye. In a second method, the pattern is presented to the subject using an organic LED device. The LED display provides greater contrast and higher luminance signals that generate large signals and reduced noise. Recordings using both methods show a difference between the schizophrenia and control populations.

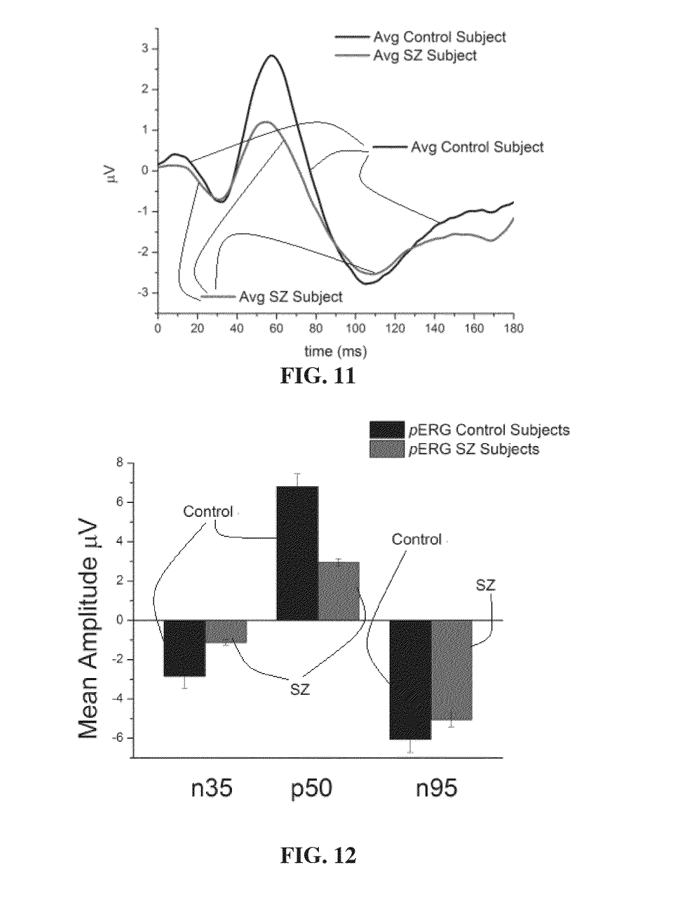

FIG. 10 illustrates the differences noted using the CRT device. To eliminate background noise, a digital filter can be applied to smooth the traces, as shown in FIG. 11. FIG. 11 illustrates pERG recordings showing lower amplitude responses for schizophrenia subjects.

FIG. 12 illustrates mean and SE for pERG components N35, P50, N95. All three response components are smaller in schizophrenia subjects compared to controls with emphasis on the differences in the P50 component.

### The Scotopic ERG

The Scotopic ERG response is associated with the rods and is recorded using a Ganzfeld LED stimulator after the subject has had a period of dark adaptation. Low-level light stimulation is used to evoke the Scotopic ERG. This response exhibits little difference when comparing the schizophrenia patients and controls.

Data from schizophrenic patients can show that the pERG is characteristically different than that recorded from normal controls. When comparing the schizophrenic pERG with the mouse pERG, recorded from an isolated, perfused retina preparation, the mouse pERG looks very much like the control human subjects. When the retina is perfused with an antagonist that eliminates the contribution of NMDA receptors to the pERG, the resulting waveform looks strikingly similar to the pERG observed in schizophrenic patients.

One example can be configured to use the pERG to study human patients with schizophrenia and compare them with age-matched and gender-matched controls. Cognitive functions of a human subject can be evaluated through testing procedures and experiments in mice can show how NMDARs contribute to the pERG response.

Mouse data can refine the pERG stimulation parameters to optimize for the NMDA receptor contribution and modify accordingly the visual stimulus used for human subjects.

Deficiencies in NMDA receptor function in the central nervous system play a role in the etiology of psychiatric disorders, with special emphasis on the genesis of schizophrenia. Retinal function can provide a window into the brain and generate insights into the etiology of schizophrenia. Recordings of the pERG can demonstrate significant differences between the pERG of patients with schizophrenia compared those of control subjects. The pERG recorded from mouse retina, after blocking NMDA receptors, bears a similarity to the pERG of patients diagnosed with schizophrenia.

According to one hypothesis, the hypofunction of NMDA receptors associated with schizophrenia can be observed in recordings from the corneal surface of the eye in the form of pERG.

NMDA receptor hypofunction results in an altered pERG in patients with schizophrenia which can be compared to normal controls. Since the pERG arises from inner retinal synapses that are partially mediated by NMDA receptors, it is predicted that the pERG can reveal characteristic differences between schizophrenic patients and controls.

The contribution of NMDA receptors to generation of the pERG, using an isolated mouse retina model, allows for pharmacological and genetic manipulations not possible in human subjects. By using well-characterized specific antagonists, NMDA receptor and other components of the pERG can be isolated. The visual stimulus can be tailored to increase or maximize the NMDA receptor component of the pERG; modifications to the visual stimulus can be used in both mice and human subjects.

One example considers whether the NMDA receptor deficiency hypothesis of schizophrenia can be evaluated directly through a strategy that relies on recording light-evoked signals from the surface of the cornea in human subjects.

Findings using pERG, carried out in both mice and humans, can raise the possibility of more directly testing the NMDA receptor hypothesis of schizophrenia by studying this disorder through signals generated by the retina, a part of the central nervous system whose synaptic network expresses NMDA receptors identical to those found in the brain.

It has been noted that chlorpromazine is effective for relaxing patients for surgery and for treating schizophrenic patients. A class of drugs known as phenothiazines is part of one therapy for treating schizophrenic patients. Phenothiazines are competitive antagonists against the D2 class of dopamine receptors. Schizophrenia is a disease in which excessive dopamine is released. It has also been reported that the use of PCP or “angel dust” produces symptoms similar to those of schizophrenia in normal subjects, causing emergency room visits for acute psychotic episodes. These reports foreshadowed the understanding that glutamate is a major excitatory neurotransmitter of the central nervous system. With appreciation that glutamate was the main CNS neurotransmitter for excitation, PCP and the phencyclidine drug class were found to antagonize a subclass of glutamate receptors identified as NMDA receptors because of their sensitivity to the glutamate agonist N-Methyl-D-Aspartate.

NMDA receptors are distributed throughout the brain, retina and spinal cord and play a role in learning, memory and cognitive functions. In addition, other drugs have been discovered, including ketamine and MK-801 that could induce schizophrenic-like symptoms by acting as NMDA receptor antagonists.

NMDA receptors are different from other types of fast-acting glutamate receptors, such as the AMPA or KA receptors (also named after agonists that selectively bind to them). Whereas glutamate binding alone can activate AMPA and KA receptors, in order for glutamate to properly gate the NMDA receptor, a second coagonist must be present at a different binding site than that for glutamate. When the need for a coagonist was first discovered for NMDA receptors, it was initially identified as glycine and it was presumed that tissue levels of glycine were sufficient to continuously saturate this site, rendering the coagonist site of questionable significance. However, shortly after discovery of a coagonist requirement for NMDAR channel gating, the amino acid D-serine was also discovered to be an effective coagonist. D-serine levels in the brain proved to be fairly high, particularly in the brain regions with higher expression of NMDA receptors (cerebral cortex, hippocampus). Shortly after this discovery, the enzyme which synthesizes D-serine from L-serine was identified (serine racemase) and its distribution was found, first in astrocytes and later more broadly distributed in neurons.

D-serine is present in the vertebrate retina. This coagonist serves as the main if not the only NMDA receptor coagonist for light-evoked activation of these receptors.

Two colonies of mice can be established which, due to knockouts/mutations of regulatory genes controlling D-serine synthesis and degradation, have either an excess or deficiency in D-serine. In characterizing these mice, consider the importance of D-serine in determining NMDA receptor function. Importantly, some patients diagnosed with schizophrenia have a deficiency of D-serine and D-serine therapy improves schizophrenic symptoms, adding support to the NMDA receptor hypofunction hypothesis of the disease. In addition, many of the genetic risk factors for schizophrenia, such as the D-Amino Acid Oxidase-Activating Gene (G72) target D-serine regulatory function. Thus one can currently make a compelling case that not only is the NMDA receptor hypofunction hypothesis attractive, but it is possible that NMDAR hypofunction found in schizophrenics reflects an abnormality in the regulation of the coagonist.

These and other observations have led to the NMDA receptor hypofunction hypothesis of schizophrenia. The failures of phenothiazines to effectively treat the full spectrum of schizophrenic symptoms (phenothiazines are more effective in treating positive symptoms such as hallucinations) and the punishing nature of the side effects these drugs have helped to make the NMDA receptor hypothesis of the disease a compelling explanation, though few drugs are yet available to treat with this strategy in mind. Nevertheless, many psychiatrists have argued that the NMDA receptor hypothesis of schizophrenia is far more attractive than the dopamine hypothesis and can itself account for exaggerated dopamine release in the brain.

The NMDA receptor hypothesis of schizophrenia is compelling. There is currently no known way of directly evaluating the existence of a deficiency in NMDA receptor function in human patients. Although evoked responses from EEG recordings can be correlated with schizophrenia, these measurements do not provide an unambiguous measurement of an NMDA receptor signal. Thus, any method that provides improvements in the ambiguity of determining if schizophrenic patients are deficient in NMDA receptor function could be pivotal as an improved means of studying this complex and debilitating illness.

According to one example of the present subject matter, a component of the ERG (or pERG) represents a biomarker that can be used in the detection of deficiencies in NMDA receptors function and prove useful as a tool for classifying and studying schizophrenia and other closely related psychiatric illnesses.

An example of the presents subject matter is directed to the study of schizophrenia through analysis of well characterized signals generated by the retina, especially those signals generated at the first level of visual processing in which NMDA receptors are involved and can be recorded through light-activation of the retinal network. The glutamatergic communication between the bipolar cells and ganglion cells of the retina is the first synaptic site at which a robust NMDAR signal can be detected. The pERG, generated by a unique visual stimulus presentation (such as a checkerboard pattern which shifts back and forth by a single square of the image at a rate of about once every two seconds), reflects a signal from retinal ganglion cells. A remarkable similarity is evident in the pERG responses of mice in which NMDA responses are blocked, and the human pERG responses of diagnosed schizophrenic patients. A pERG recording from an isolated, perfused mouse retina illustrates the effects of blocking NMDA receptors using a NMDAR competitive antagonist (D-AP7). Note that the major change in the response is a shift from near co-equal positive and negative response components to one in which the positive component was reduced, while the negative component was enhanced (these components are referred to as the P1 and N2 components in mouse retina and the P50 and N95 in humans, based on latency measurements; the pERG of the mouse retina is much slower than that of the human primarily because of the abundance of rods in the mouse; the bathing temperature for the experiments can be 30 degrees Centigrade and very little additional changes are observed at 37 degrees Centigrade where additional experiments can be carried out). A strikingly similar difference is apparent when pERGs from schizophrenic patients are compared to those of normal subjects. The data of FIG. 3 represent average pERG responses from four schizophrenic patients (right and left eyes averaged together) and 3 controls provided by one study. A smooth trace for each record can be obtained with a low pass filter of the signal-averaged data using a Savitzky-Golay algorithm with k=2 and a time limit of 26 ms (125 time steps).

Experiments can be conducted to analyze components of human electroretinograms that are significantly altered by NMDA receptor hypofunction, for example in subjects with schizophrenia. The pERG isolates the responses of inner retinal neurons, that is, those most likely to show NMDAR-dependent differences between normal subjects and those with NMDAR hypofunction. pERG is generated by retinal ganglion cells, because the response is abolished if ganglion cells are absent and is useful for evaluating patients with ganglion cell disease, including glaucoma, diabetic retinopathy and vascular insults to the inner retinal circulation.

Extensive experiments with amphibian and mammalian in vitro retinas can demonstrate the presence of NMDA receptors at inner retinal synapses. Pharmacologically blocking either glutamate binding or coagonist (D-serine) binding depresses a significant component of light-evoked synaptic activity in single retinal ganglion cells and that genetic deletion of serine racemase, which leads to depletion of the co-agonist D-serine, abolishes light-evoked NMDA receptor currents in retinal ganglion cells. The robustness of the NMDA receptor contribution measured at individual synapses suggests that an appropriate luminance stimulus delivered to the entire retina can generate a coordinated field response that also contains a strong NMDA receptor component. Accordingly, a field potential recording of light-evoked retinal activity, the proximal negative field potential (PNFP), is reduced by degrading NMDA receptor coagonist via enzymatic destruction. Experiments can indicate that the response of isolated mouse retina to pERG stimuli also contains a significant NMDA receptor contribution (FIG. 3). The results indicate that the externally recorded human pERG should also contain a measurable NMDA receptor component. Therefore, consider using pERG measurements to reveal NMDA receptor-dependent differences in retinal function between normal subjects and subjects with schizophrenia. Observing such differences supports a role for the NMDA receptor hypofunction hypothesis of schizophrenia.

The pERG method is suitable for monitoring inner retina function in human and animal subjects. The pERG is recorded from a corneal electrode during presentation of black-and-white visual stimuli (checkerboards or stripes) that reverse contrast at regular intervals. Due to the center-surround receptive field structure of retinal ganglion cells, appropriately designed visual stimuli cause outer retina responses to cancel out at the corneal electrode, leaving intact the responses of neural elements of the inner retina, which generally give a signal that is twice the frequency of the rate of stimulus change. The pERG can be used to monitor diseases of the inner retina such as glaucoma, optic neuritis, and ischemic optic neuropathy. The pERG can be performed under nonthreatening light-adapted conditions, requires no patient training, and is not susceptible to problems of motivation or the patient's clinical state.

pERG recordings can be obtained according to modified ISCEV standards on an Espion E3 system (Diagnosys LLC). Recordings can be made with corneal DTL electrodes, with the reference electrode at the lateral canthus and the ground electrode on the earlobe. A patient can be tested for scotopic, stationary flash ERG, dark-adapted rod ERG and the pERG. Because of its unique function as a signal from retinal ganglion cells, the pERG can be the primary focus of studies.

According to one example, the photopic pERG stimulus consists of a reversing black and white checkerboard target presented on a CRT at a distance of 570 mm from the subject. Testing can be performed binocularly with the subject undilated and wearing optimized corrective glasses based on the previous eye exam. In one example, data is collected and averaged in response to 150 separate stimulus presentations. The N35, P50, and N95 can be automatically marked by the software and the N95:P50 ratio can be calculated manually and compared to normative data.

In one example, pERG stimuli is provided to the isolated, perfused mouse retinas. First, plan to use the mouse retina to modify parameters of the standard pERG stimulus (currently a checkerboard pattern) to optimize isolation of the NMDA component of the electrophysiological response. Second, treat mice chronically and acutely with antipsychotic drugs, to test for effects on the mouse pERG.

The human clinical pERG can be set up and collected so that waveforms match the ISCEV recommendations of 2-4 microvolt amplitudes for P50 and N95 components, with a P50 latency to peak around 50 ms. Light stimulation usually activates both AMPA/KA and NMDA receptors. In order to isolate or enhance the NMDA receptor component, a suitable stimulation can be selected for pERG. The stimulus can be tailored to maximize the NMDA component. This can include modulating the level of light-evoked D-serine release in isolated retinas.

According to various examples, the stimulus can be selected to vary the pERG contrast or other parameters to optimize NMDA response and thereby confer more sensitivity to comparisons of subjects with and without schizophrenia. In various examples, the stimulus can include different images and patterns (such as horizontal bars, vertical bars, different colors, frequencies, and light intensities). The stimulation image and pattern can be selected to yield increased signal differences.

The pERG checkerboard stimulus parameters include contrast, background illumination, checker size (spatial frequency), and presentation rate. Time constraints in the ERG clinic, subject comfort, and inability to manipulate NMDA receptors in a human subject can make it unwieldy and time-consuming to attempt to optimize the pERG-evoked NMDA component using human subjects. One example includes performing pERG optimization experiments in the isolated, perfused mouse retina. pERG stimuli elicit a robust response in mouse retina, and blocking NMDA receptors with specific antagonists leads to profound changes in the pERG waveform.

A procedure carried out on mice can be as follows: Isolated, live retinas can be from wild-type mice. Retinas can be maintained in a perfusion chamber having continuous perfusion at a rate >2 ml per minute. This allows for vigorous, steady state responses to light stimulation for many hours. Retinas can be mounted in chambers that allow electrophysiological recording of field potentials, while beveled microelectrodes can be used to penetrate the retina; a built-in electrode recording system allows continuous monitoring of the transretinal ERG, with a relatively large pERG when the appropriate stimulus is presented. Light stimulation can be provided by computer-generated images projected through a digital projector and optically channeled through microscope objectives onto the preparation. Visual stimuli can be presented on the computer screen and on an overlay through the optics of the microscope which can be detected with a CCD camera. This arrangement provides mechanical and electrical stability, which evokes large amplitude pERG signals; fine control of light stimulus parameters are available through the computer software used to generate the images (Visionegg). This method also provides high quality images projected directly onto the retina without optical distortion from cornea and lens. This preparation also has the advantage of being free from the effects of anesthesia and provides a means for rapidly introducing pharmacological agents, by adding them to the bathing solution.

A pERG stimulus can be presented while recording the transretinal ERG. Depth penetration with beveled micropipettes can allow determining the depth profile of the pERG and validate it as a signal of the inner retina. Visual stimulation software can provide control to the in vitro pERG stimulus parameters.

In one example, systematically varied parameters include the contrast, spatial frequency (checker size), background illumination, and presentation rate of the checkerboard stimulus while recording the pERG from isolated mouse retina. Results can indicate that blocking NMDA receptors reduces the P1 component amplitude of the pERG while increasing N2. In the interests of time and minimizing exposure to drugs, some examples can be carried out while blocking NMDA receptors, using D-AP7, to manipulate the stimulus conditions that maximize P1 and minimize N2. In one example, the same parameter set is repeated in the absence of the NMDA receptor antagonist, to confirm that there is a difference between the first conditions and the second conditions. These parameters can be used for the human pERG recordings with the objective of maximizing (or increasing) differences in observed retinal NMDA receptor function between schizophrenic and non-schizophrenic subjects.

In one example, human pERG results are associated with subjects taking various antipsychotic medications. Consider whether the differences evident in the pERG recordings are due to the actions of antipsychotic medication. While some neuroleptic drugs specifically target dopaminergic systems, it is possibly they have indirect effects on NMDA receptor function or on retinal function in general. In some examples, data corresponds to the pERG in vitro from retinas of mice that have received antipsychotic agents either chronically or acutely, and compare the pERG responses to those of non-medicated animals.

FIG. 3 shows pERG recorded from an acutely isolated mouse retina under both control conditions and in the presence of the specific NMDA receptor antagonist D-AP7. Blockade of NMDA receptor function reduces the amplitude of the P1, the major positive-going component and increases the amplitude of the N2, the major negative-going component of the pERG. pERG stimulus parameters can be set to maximize the response (spatial frequency 0.05 cycles/degree, contrast of 100%, temporal frequency 1 Hz). The data corresponds to maintaining the acutely isolated mouse retina in vitro, presenting light stimuli, and obtaining electrophysiological recordings.

One example includes comparing electroretinography to reveal biomarkers of schizophrenia and NMDAR hypofunction. Retinal biomarkers can be used to identify at-risk individuals and might also be effective and efficient in monitoring therapeutic improvement associated with treatments for schizophrenia—especially treatments designed to target glutamate signaling in the brain. This can include comparing ERGs from participants with and without schizophrenia to evaluate group differences in ERG signals. Results can also be compared by conducting clinical and behavioral assessments, cognitive testing, blood draws to measure plasma levels a D-serine (a modulator of NMDA receptors) and eye exams (fundoscopy with photography of optic nerve and macula).

In one example, a processor executes an algorithm to select one of a plurality of classifications for a subject based on analysis of the ERG signal. The classifications can be correlated with changes in latency or amplitude of a feature of the signal. Classifications can be correlated with varying degrees of hypofunction or different neurological conditions.

The visual stimulus provided to the subject can be tailored to emphasize or enhance a measurable difference. In addition, the visual stimulus can be tailored to avoid or reduce artifacts. Some examples of stimulus parameters that can be selected can include light intensity, frequency, pattern (checkerboards, bars, alignment of features), and intensity contrast.

In one example, the processor executes an analysis algorithm using data expressed in the time domain. In this context, the signal is characterized by an amplitude and a time, and as such, parameters for evaluation in terms of classification include signal amplitude and time (or latency).

In one example, the processor executes an analysis algorithm using data expressed in the frequency domain. In this context, the signal is characterized by an amplitude and a frequency, and as such, parameters for evaluation include spectral content, amplitude, and area under a curve. These parameters can be evaluated and compared in order to reach conclusions as to the subject's neurological condition.

The processor can be configured to execute an algorithm to evaluate the signal using a neural network, an expert system, or artificial intelligence. In one example, an independent component analysis (ICA) is implemented and the results are used in determining a classification.

In various examples, the present subject matter evaluates a signal based on a rate of change. For example, a rise time (expressed as the time between 10% and 90%) provides a measure of neurological condition. Other parameters can also be evaluated, including a peak level, a floor or base level, variations, standard deviation, mean value, or other values. For example, a difference in magnitude (increase or decrease) of 2/3 can be correlated with a particular neurological condition.

According to one example, the present subject matter considers how signals generated by retinal cells in the eye can indicate schizophrenia and related psychiatric disorders. The pathophysiology of schizophrenia depends on numerous polygenetic and epigenetic factors, at least one of which is an alteration in glutamatergic neurotransmission. A refined version of an electroretinogram can serve as a biomarker for glutamatergic synaptic processing in the nervous system. In mice and human subjects, a pERG can be correlated with an NMDA receptor-mediated component. The pERG reflects the first synapse in retina processing that relies on light-activated NMDA and AMPA receptors of ganglion cells. NMDA receptor hypofunction in the nervous system can be correlated with pERG as a biomarker for evaluating glutamatergic synaptic transmission throughout the nervous system. The pERG reflects light-evoked ganglion cell activity generated by both AMPA and NMDA receptors and associated with a glutamatergic synaptic process.

One example of the present subject matter can be configured for evaluating drugs that act by altering glutamatergic neurotransmission. Such drugs include those tailored for treating mental disorders, such as a GlyT1 inhibitor, an mGluR2/3 agonist, and an mGluR5 antagonist.

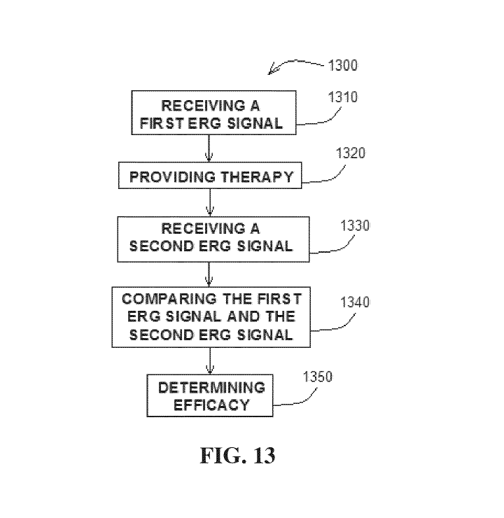

FIG. 13 illustrates a flow chart of method 1300, according to one example. At 1310, method 1300 includes receiving a first ERG signal corresponding to light evoked activity. The ERG signal is based on a sensor affixed to tissue of a subject. At 1320, method 1300 includes providing therapy to the subject after having received the first ERG signal. The therapy is configured to treat at least one of a neurological condition and a developmental etiology. At 1330, method 1300 includes, after providing therapy, receiving a second ERG signal for the subject. The second ERG signal corresponds to light evoked activity. At 1340, method 1300 includes comparing the first ERG signal and the second ERG signal. At 1350, method 1300 includes determining efficacy of the therapy based on the comparison.

Method 1300 can be tailored to evaluate a therapy or to evaluate progression of a subject. As noted in other portions of this document, the stimulation can include a predetermined geometric pattern of light. In addition, comparing the first and second ERG signals can include comparing an amplitude or comparing a time difference.

The pERG is attenuated in schizophrenic patients. Other ERG signals can also be differentiated along the line of demarcation between patients diagnosed with schizophrenia and the control group.

### Various Notes & Examples

In addition to schizophrenia, other neurological or developmental etiologies can also be evaluated using an example of the present subject matter. For example, encephalitis can be diagnosed using a suitable reference for comparison with measured data. In other examples, the present subject matter can be configured to diagnose conditions such as fetal alcohol syndrome, Alzheimer's, and autism. In addition, a genetic deficiency in certain enzymes can be diagnosed.

One example can be configured to evaluate drug efficacy. Research directed to drug efficacy can include implementing a method in which a first ERG signal is generated, therapy is delivered after the first ERG data is generated, and a second ERG data is generated after therapy. A conclusion as to treatment efficacy can be reached based on a comparison of the first ERG and the second ERG.

In one example, a method can be implemented to distinguish bipolar disease from other neurological conditions.

The inventor has recognized that synaptic function in the retina, which is physiologically and developmentally part of the central nervous system (CNS), mirrors that found in other parts of the CNS.

Disturbances in CNS synaptic activity are present in retinal synaptic systems. Schizophrenia reflects hypofunction of NMDA receptors, a major subtype of ionotropic glutamate receptor.

In one example, the retina serves as a window into deficits in glutamate-mediated neurotransmission in the brain that may contribute to the development of schizophrenia.

The present subject matter entails using a retinal signal of NMDA receptor activation for classifying and studying a disease as a deficiency in NMDA receptor function. The activation of retinal ganglion cells can be viewed as the first step in the visual pathway in which NMDA receptors are activated. Signals recorded from the human eye, such as using the pERG can serve as a biomarker for NMDA receptor hypofunction and schizophrenia. Such a biomarker can be correlated with a NMDA hypofunction hypothesis of schizophrenia, and thus facilitate identification of individuals at risk for the illness, and evaluate the therapeutic improvement of treatments.

Data can demonstrate the feasibility of this strategy in the mouse retina, in which the NMDA receptor component is correlated with the pERG. Human data can also show a correlation between ERG measurements from subjects with and without schizophrenia.

Methods currently available do not directly evaluate one of the main theories about schizophrenia—that it is a disease of a subtype glutamate receptor, known as the NMDA receptor. The present subject matter measures the activity of these receptors. Recordings from schizophrenia shows a measurable difference.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system comprising:
a display configured to provide a controlled light stimulus;
a sensor having an electrode configured for coupling to tissue of a subject, the sensor configured to generate an output signal corresponding to an electroretinogram based on light evoked activity associated with synaptic processing, the light evoked activity in response to an image on the display; and
a processor coupled to the sensor and coupled to the display, the processor configured to classify a neurological condition associated with the subject, the classification based on the output signal.

2. The system of claim 1 wherein the processor is configured to classify based on a frequency domain representation of the output signal.

3. The system of claim 1 wherein the controlled light stimulus includes a red flash on a blue background.

4. The system of claim 1 wherein the classification is based on a peak amplitude of the output signal.

5. The system of claim 1 wherein the controlled light stimulus includes a period of light adaptation followed by a light flash.

6. The system of claim 1 wherein the classification is based on a time delay in a feature of the output signal.

7. The system of claim 1 wherein the controlled light stimulus includes light stimulation at a frequency of approximately 30 Hz.

8. The system of claim 1 wherein the processor includes a filter.

9. The system of claim 1 wherein the controlled light stimulus includes an alternated checkerboard pattern.

10. The system of claim 1 wherein the display includes a LED device.

11. The system of claim 1 wherein the sensor includes a contact surface.

12. A method comprising
receiving an electroretinogram (ERG) signal from a surface sensor, the ERG signal corresponding to tissue of interest for a subject in response to visible light stimulation;
comparing the ERG signal and a reference; and
determining a neurological condition of the subject based on the comparison.

13. The method of claim 12 wherein receiving the ERG signal includes providing stimulation using a geometric pattern.

14. The method of claim 12 wherein receiving the ERG signal includes receiving time domain data.

15. The method of claim 12 wherein comparing the ERG signal with the reference includes comparing an amplitude.

16. The method of claim 12 wherein comparing the ERG signal includes processing the signal.

17. The method of claim 16 wherein processing includes filtering.

18. The method of claim 12 wherein classifying includes diagnosing schizophrenia.

19. The method of claim 12 wherein classifying includes diagnosing a neurological condition.

20. A method comprising:
receiving an electroretinogram (ERG) signal corresponding to a subject, the signal in response to stimulus including red light on a blue background, the signal based on a sensor coupled to tissue of the subject;
identifying a positive response in the signal; and
upon detecting absence of a negative response in the signal following the positive response, making a determination concerning a neurological condition.

21. The method of claim 20 wherein receiving the ERG signal includes providing the stimulus having a light intensity of greater than 5 candela-second per square meter.

22. The method of claim 20 wherein detecting absence includes monitoring an amplitude of the signal.
